(19) United States
(12) Patent Application Publication
(10) Pub. No.: US 2004/0260019 A1
(43) Pub. Date: Dec. 23, 2004

(54) Zero length cross-linking of proteins and related compounds

(76) Inventors: Harvey Kaplan, Ottawa (CA); Mary Alice Hefford, Ottawa (CA); Brigitte Leanne Simons, Ottawa (CA)

Correspondence Address: Adrian M Kaplan, Dimock Stratton, 20 Queen Street West, Suite 3202 Box 10, Toronto, ON, M5H 3R3, CA

(21) Appl. No.: 10/487,433

(22) PCT Filed: Aug. 19, 2002
(86) PCT No.: PCT/CA02/01277

(51) Int. Cl. C08G 63/48; C08G 63/91

(52) U.S. Cl. 525/54.1

(57) ABSTRACT

The invention relates to a method of cross-linking poly(amino acid) compounds (e.g. proteins, polypeptides, protein polymers, etc.) to each other or attaching such compounds to polyamino or polycarboxyl compounds. The method involves lyophilizing a solution of at least one poly(amino acid) compound, or at least one poly(amino acid) compound and a polyamino or polycarboxyl compound, maintaining the lyophilized solid under vacuum, heating the lyophilized solid under vacuum to an elevated temperature effective to cause cross-linking without denaturing of the poly(amino acid) compounds, and cooling the product and releasing the vacuum. The method produces cross-linking without the use of activating compounds or cross-linking molecules. The invention also relates to novel cross-linked products.

### TECHNICAL FIELD

This invention relates to the cross-linking of molecules of proteins or other poly(amino acid) compounds to each other, or to polyamino or polycarboxyl compounds, and more particularly, although not exclusively, to the cross-linking or attachment of molecules of complex proteins in such a way that the proteins retain biological or biochemical activity.

### BACKGROUND ART

Proteins have been subjected to reactions that cause chemical cross-linking of the protein molecules in order to create cross-linked products that may have advantages over the native proteins themselves. For example, cross-linked hemoglobin is used as a blood substitute. Furthermore, there are many other potential applications of cross-linked protein products of this kind.

The conventional processes for cross-linking protein molecules generally involve the reaction of protein monomers with short bi-functional chemical modifying agents in aqueous solution. For example, Lundblad (Lundblad R., 1994, Techniques in Protein Modification. CRC Press, Boca Raton, Fla., USA) has disclosed a process involving activation of carboxyl groups of a protein with a water-soluble carbodiimide compound followed by coupling with an amino group of an adjacent protein molecule to form a stable amide bond. However, there are disadvantages to such procedures. For example, the chemical modifying agents are incorporated into the resulting cross-linked product, and this may change or destroy the chemical or biological activity of the original proteins. In addition, undesirable antigenic properties may be introduced into the cross-linked protein product by the modifying agent. A further disadvantage is that only proteins soluble in aqueous media are in general suitable for reactions of this kind and this is not the case for all proteins. Also, current methods for cross-linking protein molecules require relatively large amounts of protein.

U.S. Pat. No. 4,703,108 which issued to Silver et al. on Oct. 27, 1987 discloses a process for preparing collagen-based matrices in sponge or sheet form in which a collagen-based material is freeze dried to form a collagen-based sponge which is contacted with a cross-linking agent (a carbodiimide or succinimidyl active ester) and reacted to form an intermediate collagen-based matrix that is then subjected to severe dehydration to form a collagen-based matrix in sponge or sheet form.

A process that the authors call “dehydrothermal crosslinking” as applied to collagen has been described in Preparation and Characterization of Porous Crosslinked Collagenous Matrices Containing Bioavailable Chondriontin Sulphate: Piper, J. S. et al. 1999, Biomaterials 20, 847-858; Effect of Chemical modification on the susceptibility of collagen to proteolysis. II. Dehydrothermal Crosslinking: Gorham, S. D. et. al. 1992, Int. J Biol Macromol. 14, 129-138; and Evaluating Collagen Crosslinking Techniques: Weedock, K. et al.,1983 Biomaterials, 11, 293-318). Collagen is an insoluble highly interlocked fibrous structure that is strengthened by natural cross-links involving the amino function of lysyl residues. The dehydrothermal cross-linking process applies only to collagen because of its specific structure and natural cross-linking tendency and because it is promoting the formation of lysino-alanine crosslinks by a dehydration process. No evidence is provided that that peptide bond formation is taking place or that this cross-linking process is applicable to proteins other than collagen.

There is therefore a need for an improved method of cross-linking proteins, and related compounds having amino and carboxyl groups, of more general applicability; particularly a method that enables heat sensitive reactive compounds to retain or enhance the biological, biochemical or other activity of the original reactants.

### DISCLOSURE OF THE INVENTION

An object of the present invention, at least in its preferred forms, is to provide a method for so-called “zero-length” (i.e. linker-free) cross-linking of proteins and related compounds.

Another object of the invention, at least in preferred forms, is to provide a method of reacting protein molecules to each other or to molecules of polyamino or polycarboxyl compounds.

Another object of the invention, at least in preferred forms, is to provide cross-linked protein products having desirable commercial uses.

Another object of the invention, at least in preferred forms, is to provide a method of cross-linking biologically active proteins in a way that retains substantially the same or improved biological activity in the cross-linked products.

Yet another object of the invention, at least in its preferred forms, is to provide a process of cross-linking proteins that is effective, as desired, on relatively small amounts or alternatively large amounts of protein.

A still further object of the invention, at least in preferred forms, is to provide a process for directly reacting one or more proteins with polycarboxyl or polyamino compounds.

The present invention is based on the finding that molecules poly(amino acid) compounds (e.g. polypeptides and proteins) having unreacted amino acid and carboxylic acid radicals may be caused to cross-link together directly, i.e. without the intervention of other compounds, if the compounds are obtained in solid form by lyophilization and are then heated in vacuo. Thus, in a broad sense, the present invention provides a method of forming a covalent bond between a molecule having at least one amino group and a molecule having at least one carboxyl group, wherein a solution containing the molecules is formed, the solution is lyophilized to form a lyophilized solid, the lyophilized solid is maintained under vacuum, the lyophilized solid maintained under vacuum is heated to an elevated temperature effective to form said covalent bond, and the vacuum is released.

More speficially, according to one aspect of the invention, there is provided a method of cross-linking molecules of poly(amino acid) compounds together or attaching such molecules to molecules of a polyamino or polycarboxyl compound, which comprises lyophilizing a solution containing molecules of at least one poly(amino acid) compound, or at least one poly(amino acid) compound and a polyamino or polycarboxyl compound, thereby producing a lyophilized solid, maintaining the lyophilized solid under vacuum, heating the lyophilized solid maintained under vacuum to an elevated temperature effective to cause cross-linking or attachment of the molecules, thereby producing a reaction mixture comprising at least one cross-linked poly(amino acid) compound or at least one poly(amino acid) compound attached to the polyamino or polycarboxl compound, and cooling the reaction mixture and releasing the vacuum.

According to another aspect of the invention, there is provided a product having cross-linked molecules of at least one poly(amino acid) compound, or molecules of at least one poly(amino acid) compound attached to molecules of a polyamino or polycarboxyl compound, produced by the method defined above.

According to yet another aspect of the invention, there is provided a dimer of RNase A in which residues of RNase monomer molecules are directly covalently cross-linked via peptide bonds. The dimer has a molecular weight of about 28.8 kDa.

According to yet another aspect of the invention, there is provided a reagent for a Western Blot test comprising a covalently cross-linked product containing residues of at least one antibody protein capable of bonding with a target antigen, at least one enzyme detector protein, and a polyamine or polycarboxyl compound, said residues being cross-linked via direct peptide bonds.

According to yet another aspect of the invention, there is provided a process of directly attaching at least one protein molecule to a polycarboxyl or polyamino compound, which comprises lyophilizing a solution of at least one protein and at least one polyamino or polycarboxyl compound, thereby producing a lyophilized solid; maintaining the lyophilized solid under vacuum; heating the lyophilized solid maintained under vacuum to an elevated temperature effective to cause cross-linking of the protein and the polyamino or polycarboxyl compound, thereby producing a product comprising a covalently cross-linked protein and polyamino or polycarboxyl compound; cooling the product and releasing the vacuum to produce a reaction mixture; and, if desired, isolating a cross-linked product from the reaction mixture.

According to still another aspect of the invention, there is provided a method of cross-linking protein molecules, which comprises lyophilizing a solution of at least one protein thereby producing a lyophilized solid, maintaining the lyophilized solid under vacuum, heating the lyophilized solid maintained under vacuum to an elevated temperature effective to cause cross-linking of said at least one protein, thereby producing a product comprising at least one cross-linked protein; and cooling the product and releasing the vacuum.

If one or more of the proteins is a complex protein having a native structure and a biological or biochemical activity, the solution subjected to lyophilizing should preferably have a pH value that allows the protein to retain its native structure and activity.

The poly(amino acid) compound (or one of the poly(amino acid) compounds, if a mixture is used) employed as a starting material in the above reaction may be a cross-linked product of an earlier reaction of the same kind, thus resulting in an even larger cross-linked product molecule.

The invention also relates to cross-linked poly(amino acid) products produced by the method and to novel cross-linked poly(amino acid) compounds (particularly proteins) per se.

As noted above, the invention includes a process of directly cross-linking at least one protein molecule to a molecule of a polyamino or polycarboxyl compound, which may be a protein polymer (i.e a synthetic polypeptide of considerable length). In such cases, the polyamino or polycarboxyl compounds are preferably of quite large size, e.g. having a molecular weight of at least 1000 Da, or even more than 300,000 Da (examples being those compounds having molecular weights of up to about 2000, 3000, 4000, 15000, 30,000, 70,000, 150000 or 300000 Da). Polylysine is an example of a suitable polyamino compound and polyglutamic acid is an example of a suitable polycarboxyl compound.

The above procedure may be repeated using the cross-linked protein product obtained by a similar reaction and a second protein, thereby producing a product having two proteins directly cross-linked to a polyamino or polycarboxyl compound. Further repetitions may increase the number of proteins attached to the polycarboxyl or polyamino compound. Alternatively, a single reaction may be carried out using a solution containing a polyamino or polycarboxyl compound and several proteins.

If desired in the processes described above, the polyamino or polycarboxyl compound may be attached to a solid surface, either before, during or after the cross-linking reaction, in order to produce a complex in which one or more proteins are indirectly bonded to a solid surface for use in test procedures or for other purposes. While such a compound does not dissolve in a solution, it nevertheless may come into contact with a solution containing another reactant and may therefore be regarded as forming part of the solution of reactants that is subjected to lyophilization.

The products of the invention are most commonly (but not necessarily exclusively) of two general and preferred kinds, i.e. (1) oligomers, e.g. dimers, trimers, tetramers, pentamers, etc., of one or more simple or complex proteins (or less commonly polypeptides) having direct peptides bonds formed between residues of the original molecules, and (2) reaction products of polyamino or polycarboxyl compounds and one or more simple or complex proteins (or less commonly polypeptides), having direct peptide bonds formed between the residues of the polyamino or polycarboxyl compounds and the protein (or polypeptide) molecules. The oligomers of product type (1) may be homo-oligomers (i.e. the residues are all of the same protein or polypeptide) or hetero-oligomers. The products of type (2) generally have a single residue of a polyamino or polycarboxyl compound linked to one or more protein (or polypeptide) residues which, when there are more than one, may be of the same or different kinds. Most preferably, the cross-linked products of the present invention comprise a residue of at least one complex protein.

When molecules of complex proteins react to form products of type (1), generally only two molecules link together to form dimers. Larger polymers may be produced, but usually only in small quantities and often as insoluble precipitates of limited usefulness. The extent of reaction, as well as the relative proportions of oligomeric species in the cross-linked product, is dependent on the properties of the individual protein(s) in question and can be controlled by altering the reaction conditions, viz. time, temperature, pH of lyophilization and quantity of protein, etc. The cross-linked protein products of this type may resemble dimers and other oligomers that tend to form naturally when protein molecules are present in solution by virtue of non-covalent bonds, e.g. hydrogen bonds and ionic interaction, that form between the protein molecules. Like such non-covalent oligomers, the covalently bonded oligomers formed by cross-linking of monomeric units of the present invention usually retain their original biological activity.

The conditions employed for the process of the invention may be chosen to produce mainly dimers with predominantly one direct (zero-length) cross-link between the monomer units, although dimers with two or more cross-links may occasionally be formed under some conditions. In the case of RNase A, it has been found that the dimer having one cross-link shows activity towards all common single-stranded RNA, double-stranded RNA and total RNA as substrates. The dimer is also considerably more active towards double-stranded RNA substrates than the naturally-occurring monomer, and is significantly less susceptible to inhibition by cystolic(cellular) ribonuclease inhibitor protein (cRI) than the naturally-occurring monomer.

It is of note that the reaction of the invention is carried out in the absence of reactive bi-functional cross-linking agents as conventionally used (generally small bi-functional molecules such as carbodiimide), and is normally carried out without activating agents or catalysts. The products of the present invention not only lack residues of cross-linking agents in the bonds between molecules, but may have cross-links at different positions than in known cross-linked proteins, thus producing novel cross-linked products.

The cross-linked products of the present invention will have several industrial and therapeutic applications, e.g. for the attachment of enzymes to polymers and plastics, the construction of immunotoxins, and the preparation of “magic bullet” drugs, to name but a few.

Definitions

By the term poly(amino acid) compound as used herein, we mean any compound that contains residues of amino acid molecules covalently linked together via peptide bonds. The term may include, for example, natural or synthetic proteins (both simple and complex), polypeptides, protein polymers, etc., provided the compounds have an amino or carboxyl group or groups available for the cross-linking reaction of the present invention.

By the term “protein” as used herein, we mean to include compounds that consist of one or several polypeptide chains, each of which is a polymer of a considerable number (e.g. a hundred, two hundred or more) amino acids linked by peptide bonds. Typically, proteins have molecular weights ranging from about 6000 to several million Da. The polypeptide chain(s) may undergo coiling or pleating, the nature and extent of which is referred to as the secondary structure of the protein. The coiled or pleated polypeptides may adopt a three-dimensional conformation referred to as the tertiary structure of the protein. The proteins may include both naturally-occurring products and the products of recombinant DNA or other synthetic techniques. For convenience of expression, the term “protein” may on occasion be taken to include polypeptides (i.e. short molecules that may not have defined conformation or recognized biological activity) as well as simple or complex proteins, but such uses will be apparent from the context in which they are used.

By the term “complex protein” as used herein we mean proteins having a defined conformation (e.g. a native structure of three-dimensional folding and possible internal cross-linking) and/or recognized biological activity in living organisms or biochemical activity on non-living substrates. It follows that a “simple” protein is a protein that does not have defined conformation and/or biological or biochemical activity of a complex protein, and is usually of lower molecular weight.

By the term “polypeptide” as used herein we mean all natural and synthetic poly(amino acid) compounds having molecules made up of three or more (or more preferably 10 or more) substituted or unsubstituted amino acids internally linked by peptide bonds. Generally, polypeptides have a smaller number of amino acid residues than proteins (usually less than 100 amino acid residues), and are short molecules that may not have any biological or biochemical function. Generally, the molecular weight of polypeptides is less than 10,000 Da.

The term “polyamino compound” or “polycarboxyl compound” as used herein means a compound having a plurality of unreacted amino groups, or alternatively unreacted carboxyl groups. The compounds may fall under the above definition of “protein” or “polypeptide” (i.e. polypeptide or protein polymers) or may possibly be other molecules (e.g. compounds including a chain of carbon-carbon bonds with amine or carboxylic acid substitutents). These compounds may be of considerable size (molecular weight). For example, it may be desirable to use compounds of this type that are larger in size (molecular weight) than simple or complex proteins with which they are reacted according to the present invention. This may assist separation procedures.

The term “lyophilize” as used herein means the removal of liquid from heat-sensitive materials such as proteins. A protein solution is frozen, placed under a high vacuum, and maintained in the frozen state (e.g. at a temperature below −40° C.). The low pressure generated by the vacuum causes the ice (or other solidified liquid) formed by freezing to turn from a solid to a gaseous form without passing through a liquid state. This allows the removal of liquid from the protein without otherwise disturbing its composition or characteristics. The term “freeze drying” is often used to refer to the same procedure.

The term “direct peptide bond” as used herein means a covalent bond formed between residues of two protein molecules (or between a protein molecule and a polyamino or polycarboxyl compound) formed directly from an amine group of one molecule and a carboxyl group of another molecule. There are consequently no residues of a linker compound within the bond between the two molecules. The process of forming direct peptide bonds of this kind is referred to herein as “zero-length cross-linking” because there is only a single covalent bond (a molecular chain of zero-length) between the residues of the reacting molecules.

### BRIEF DESCRIPTION OF THE DRAWINGS

FIG. 1 is a schematic representation of a zero-length cross-linking reaction according to one embodiment of the present invention.

FIG. 2A is an illustration of the cross-linking of a polyamino compound, e.g. polylysine, with an unspecified protein P;

FIG. 2B is an illustration of attachment of a polyamino compound to a surface for immobilization thereon.

FIG. 3 is an illustration of the cross-linking of a polycarboxy compound (e.g. polyglutamic acid) with an unspecified protein P;

FIG. 4A is an illustration of the formation of a polylysine complex cross-linked with an antibody and with an enzyme, the construct being suitable for a reagent used for Western Blot analysis;

FIG. 4B is an illustration similar to FIG. 4A, but with polyglutamic acid;

FIG. 5 is a reproduction of an SDS-PAGE plate showing results explained in Example 1 below;

FIG. 6 shows a gel assay of RNase activity on (i) RNase supplied by the manufacturer, (ii) isolated monomer and (iii) isolated dimer after crosslinking with by the method described in this invention;

FIG. 7 is a reproduction of an electrophoresis gel plate showing results explained in the Examples below;

FIGS. 8 to 11 and 13 are reproductions of an electrophoresis gel plate showing results explained in the Examples below; and

FIGS. 12 and 14 to 16 are graphs or traces showing experimental results described in the Examples below.

### BEST MODES FOR CARRYING OUT THE INVENTION

The present invention makes use of the previously unrecognized ability of protein molecules to cross-link together covalently, either with themselves or with molecules of other proteins or polyamino or polycarboxyl compounds, in the solid phase under vacuum at elevated temperature without the need for additional chemicals to act as activators, linkers or catalysts. Without wishing to be bound by any particular theory of operation, evidence has been obtained that the cross-linking reaction takes place by direct peptide bond formation between a protonated amino group of one protein molecule and a deprotonated carboxyl group of another protein, i.e. as by the condensation reaction as follows:

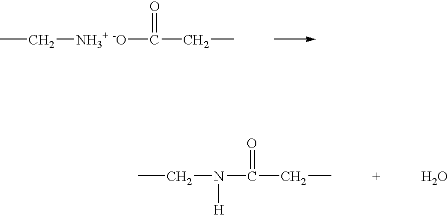

This is an energetically unfavourable reaction under aqueous conditions, so it is somewhat surprising that it takes place in mild conditions in the solid phase without catalysts or cross-linkers. However, it is believed that by carrying out the reaction under vacuum, the removal of the water by-product drives the reaction to the right and causes good yields, generally of at least 25% by weight (e.g often about 30% by weight), which is high enough for commercial acceptability. The yields attained may in fact be much higher, depending on the protein and conditions employed. Increasing the temperature of the reaction generally increases the yield of cross-linked product with temperatures between 100 and 120° C. generating the highest yields. Cross-linking has been accomplished with all of the proteins and protein mixtures employed at present. It is predicted that the reaction will take place with most or all protein molecules, so the present invention has broad applicability.

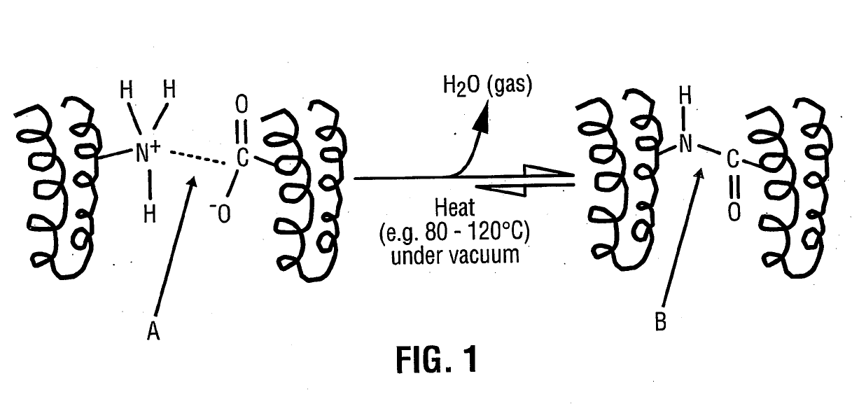

The cross-linking reaction is illustrated graphically in FIG. 1 in which the curled strands represent complex protein molecules. Again without wishing to be bound by any particular theory of operation, it is believed that direct interaction of the protein molecules before and after lyophilization is required, e.g. by the formation of salt bridges (i.e ionic bonds—shown by a dotted line and labeled A in FIG. 1) between interacting ammonium and carboxylate functions formed in appropriate conditions of pH. In the cross-linked product, the a covalent bond (shown as a solid line and labeled B in FIG. 1) is formed to replace the salt bridge. More specifically, the cross-linking reaction takes place when an amino group is protonated and a carboxyl group is deprotonated. This condition exists if the lyophilized protein or mixture of proteins is obtained by freeze drying a solution having a suitable pH. The effective pH range may vary from protein to protein, but is generally in the range of pH 4 to 10, more preferably approximately neutral to slightly alkaline (e.g. pH 6 to 9), and optimally pH 7 to 8 or pH 7 to 9. Not only do these pH values cause the desired protonation of amino groups and deprotonation of carboxyl groups of the protein molecules, but they also avoid denaturing of some proteins that may take place at higher or lower pH values. The pH of a solution of a protein or protein mixture may, of course, be modified (when necessary) by adding a suitable acid or base to the solution in a manner well known to persons skilled in the art.

Generally, the protein solutions employed in the present invention are aqueous, but solutions in other solvents or solvent mixtures may be contemplated, provided that lyophilization is possible.

If the desired reaction is the formation of an oligomer made up of the same monomer unit (homo-oligomer), the solution formed prior to lyophilization should contain just one protein. On the other hand, if an oligomer made up of two or more different monomer units (hetero-oligomer) is desired, the solution should contain more than one protein. In the latter case, several cross-linked products will normally then be produced. For example, if there are two different proteins, cross-linking will usually take place between the two proteins, but also the individual proteins will cross-link with themselves, thus forming two different homo-oligomers and a hetero-oligomer. The relative proportions of the products formed can be biased by adjusting the relative starting amounts of the different proteins and/or the reaction conditions. Separation of the desired products from the reaction mixture and from each other may be required.

Procedures for lyophilizing proteins are well known in the art and any suitable procedure may be used to form lyophilized proteins or protein mixtures for application in the present invention. Of course, the procedure employed should use conditions that are suitably mild to avoid denaturing or modification of the proteins. For operation of the present invention on a large commercial scale, techniques and equipment currently used for the freeze drying of beverages, such as coffee, may be employed for lyophilizing proteins for the present invention.

As previously noted, lyophilization (freeze drying) generally involves removal of water from a frozen solution under vacuum. The resulting solid is consequently, at the end of the lyophilization procedure, obtained under a vacuum. If the solid is already in a suitable container, therefore, the vacuum may be maintained for the cross-linking reaction of the present invention, which may then be carried out in the lyophilization vessel. Alternatively, the solid may be transferred to another container and the vacuum reapplied, if released during the transfer. For small scale operations, freeze-dried proteins or protein mixtures may be placed within a sealable container, e.g. a glass vial, and the space above the solid may be evacuated by connecting the container to a conventional vacuum pump. The container may then be sealed, e.g. by heating and pinching closed an upper section a glass vial.

The degree of vacuum employed for the present invention is not especially critical. It should, of course, be sufficient to draw off the water (which is the by-product of the cross-linking reaction) from the solid reactants, and thus help to drive the reaction in the desired direction. In general, the cross-linking reaction proceeds better as the degree of vacuum is increased, but an ultra-high vacuum need not be used. Indeed, ultra-high vacuums may have the undesirable effect of removing “essential water” from the protein, i.e. water bound to the structure and assisting with the folding or conformation of the protein. Generally, a vacuum of at most 500 milli-tor is sufficient with 50 to 10 milli-torr being preferred. If the cross-linking reaction of the present invention is attempted in the absence of a sufficient vacuum, the protein(s) often undergo breakdown, chemical modification or denaturing.

The reaction of the present invention takes place at an elevated temperature, i.e. a temperature above room temperature (i.e. above 21° C.) and preferably above ambient temperature (which may be taken to range up to 25° C. or so). A distinct heating step is therefore required or the reaction takes place too slowly (if at all). As expected, higher temperatures accelerate the cross-linking reaction, but the temperature should not be so high that denaturing or undesirable reactions take place. The maximum effective temperature varies from protein to protein, but is usually not higher than 150° C. In fact, the preferred temperature range for the present invention is 50-120° C., more preferably 80-120° C., or even 100-120° C. as noted above (although a temperature range of 70 to 100° C. may be preferred for some proteins).

The heating step may be carried out by any suitable method. For example, a container holding the lyophilized solid may be heated by incubating in a hot water bath, in an oven, or by an electric or other heater. However, care should be taken to avoid hot spots that may caused localized overheating of the lyophilized solid, and some sort of heated liquid bath or oven is preferred.

The duration of the reaction, i.e. the time for which the lyophilized solid is maintained under vacuum at the reaction temperature, may vary according to the protein(s) employed, the reaction temperature selected, and the desired extent of cross-liking. Normally, the reaction time is within the range of 1 to 24 hours, but could be as high as several days to a week if a very low reaction temperature is employed (e.g. when carrying out the reaction with a protein that is very heat-sensitive, thus requiring an unusually low reaction temperature). Longer reaction times may also be required if higher oligomers are required (oligomers containing more monomer units tend to be formed more slowly than those with fewer monomer units, e.g. dimers). Moreover, longer times may also be required if the lyophilized solid contains an excipient or other inert molecules (e.g. for reasons explained below).

It is to be noted that the reaction of the present invention (at least when used to cross-link one or more complex proteins) is carried out in the absence of cross-linking reagents, such as those conventionally used for cross-linking proteins (e.g. bifunctional, multifunctional or activating reagents), and other molecules that may be incorporated into the polymer product by covalent bonding. This has the advantage that the nature of the protein is changed as little as possible by the cross-linking reaction (so-called “zero-length cross-linking is achieved), so the natural conformation and bio-activity are generally retained, and there is no risk of adding a substance that may be bio-incompatible in systems with which the product will be used.

As noted above, however, excipients, diluents, or the like (i.e. inert compounds) may be mixed with the proteins prior to reaction. The purpose of this may be, for example, to affect the degree of cross-linking, the extent of polymerization (number of monomer units per oligomer molecule), or otherwise to modify the reaction. Excipients used in this way are generally biologically unreactive materials that do not become cross-linked with the proteins, e.g. trehalose. Usually the lyophilized solid prior to reaction contains less than 30% by weight of excipient and some excipients may be effective in very small amounts less that 0.01% by weight. Trehalose, for example, when present in the original reaction mixture, tends to replace the solvent shell around the protein molecules, thereby stabilizing the molecules, but this may isolate the molecules from eachother to some extent, thus reducing the extent of cross-linking.

After the reaction of the present invention has been carried out for a suitable length of time, the reaction mixture is cooled and the vacuum released (the vacuum may be released either before, during or after cooling commences or terminates). The reaction mixture may then be obtained and used in any desired way. In some cases, no further treatment of the reaction product may be needed, but in other cases, separation of the cross-linked product(s) from unreacted starting materials will usually be required, and it may be necessary to separate different reaction products from each other in those cases where more than one cross-linked product is produced. Any suitable method for separation of proteins may be employed for this task. For example, size exclusion chromatography and reverse phase chromatography may be employed. These and other suitable techniques are well known to persons skilled in the art.

Unreacted monomers separated from the reaction mixture of the present invention may be recycled and reacted again, if desired. Moreover, already cross-linked proteins that are either the products of the reaction of the present invention, or are obtained by other means, may be subjected to the cross-linking reaction of the present invention, thereby undergoing further cross-linking to make longer (or larger) polymers containing the same monomer units or introducing different monomer units. In this way, cross-linked protein products may be produced that contain several different protein monomer units introduced during successive reaction steps.

Proteins that may be cross-linked according to the present invention are numerous, as indicated above, but some are of particular commercial interest. For example, it is particularly desirable to obtain cross-linked forms of hemoglobin, ribonuclease, antibodies, enzymes and synthetic polyamines or polycarboxyl compounds such as polylysine and polyglutamic acid.

Specific examples include alkaline phosphatase, human growth hormone, bovine serum albumin, beta-galactosidase, trypsin, chymotrypsin, Bt-toxin, cytochrome c and de novo designed proteins MB-1 and variants (see M. Beauregard, C. Dupont, R. M. Teather and M. A. Hefford, Biotechnology 13:974-981, 1995; Matthew H. Parker and Mary Alice Hefford, Protein Engineering 10: (5) 487-496, 1997; Brigitte Simons, Dean Scholl, Terry Cyr, and Mary Alice Hefford: Protein and Peptide Letters 8(2):89-96, 2001; the disclosures of which are incorporated herein by reference).

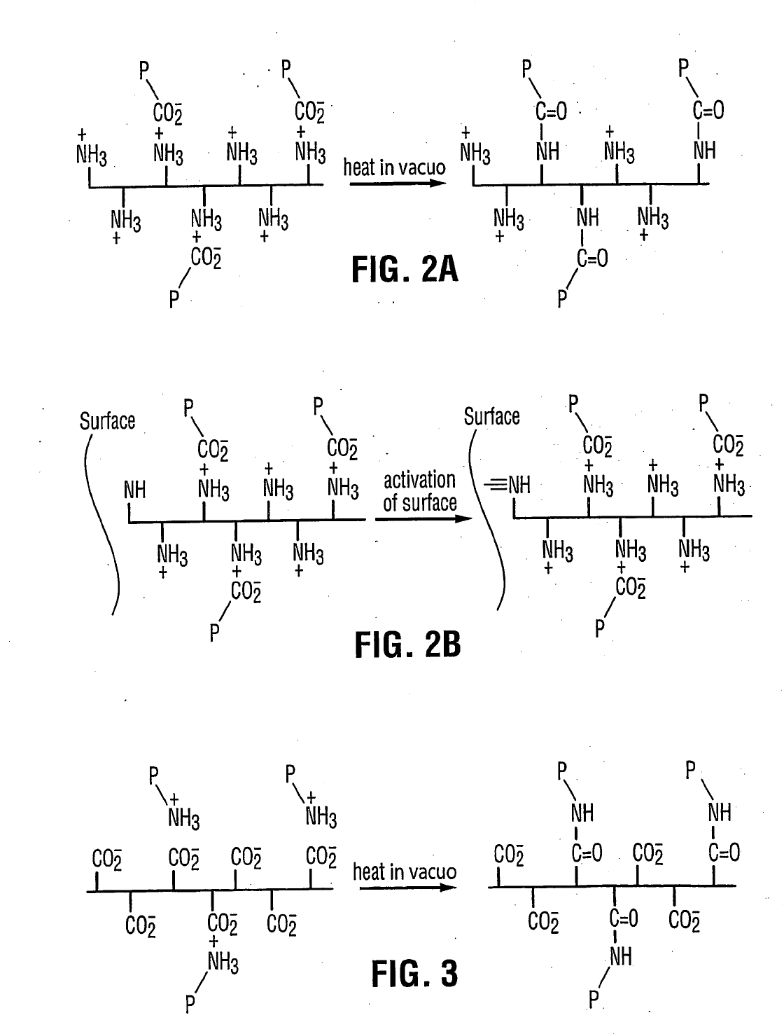

Polylysine and polyglutamic acid are commercially available protein polymer products. Polylysine (which may for example be obtained from Sigma—www.sigma.com—has free amino groups that may be reacted with carboxyl groups of simple or complex proteins (e.g. enzymes), whereas polyglutamic acid has free carboxyl groups that may be reacted with amino groups of simple or complex proteins. Compounds of this kind may be immobilized on solid surfaces. Thus, the present invention may be used to attach proteins to these compounds and to immobilize them to solid surfaces and the like by covalent attachment through the amino or carboxyl group on polylysine or polyglutaric acid. FIGS. 2A, 2B and 3 of the accompanying drawings illustrate such reactions of polylysine and polyglutamic acid according to the present invention.

FIG. 2A illustrates the in vacuo attachment of protein (1) to a polyamino (e.g. polylysine) compound. The protein P may be an enzyme or any other protein, but an enzyme is given as an example. The formula at the left hand side of the drawing represents the starting material, which is a co-lyophilized protein mixture of the enzyme and the polyamino compound. The reaction upon heating in vacuo produces a cross-linked copolymer.

FIG. 2B represents the attachment of the polyamino compound to glass upon activation of the surface prior to cross-linking. Attachment of the polyamino compound to the surface can be carried out by known procedures, actually before the cross-linking reaction of the present invention, during the cross-linking or after. The attachment may be accomplished, for example, by derivatizing the glass and then using appropriate solution chemistry to attach the polyamino or polycarboxyl compound, protein or polypeptide. This can be accomplished using the in vacuo procedure of the present invention after appropriate derivatization of the glass. The compound or protein is lyophilized with the modified glass container or on a separate substrate, such as glass beads, sealed under vacuum and incubated at elevated temperature.

FIG. 3 illustrates the in vacuo attachment of protein P (e.g. an enzyme) to a polycarboxyl compound (e.g. polyglutamic acid). The material at the left of the drawing is a co-lyophilized mixture of the polycarboxyl compound and the protein P.

Reactions of this type are of significant commercial interest. For example, in Western Blot analysis, a mixture of protein antigens is bound to a synthetic membrane (e.g. PVDF, nylon, nitrocellulose) and specific antigens are identified by the binding action of such antigens to antibodies raised against them. The antibodies are associated with an enzyme capable of changing the color of a detection compound. In such a system, a protein polymer such as polylysine may be cross-linked with both the antibody and to the enzyme in such a way that the polylysine binds several enzyme molecules for each antibody molecule (this ratio of attachment may be assured by appropriately choosing amounts of the enzyme and antibody with respect to the protein polymer for the cross-linking reactions). When such a complex becomes bound to an immobilized protein, there are several color-changing enzymes molecules per antibody-antigen complex, so the color change required for detection will be much more pronounced and therefore detectable. The blotting test can therefore be made highly sensitive, so that even very small amounts of antigen may be detected. Such situations are common, for example, in two-dimensional electrophoretic gels used to separate proteins in proteomics analysis and a reagent of such enhanced sensitivity may allow in-gel detection of individual, low abundant components of the proteome.

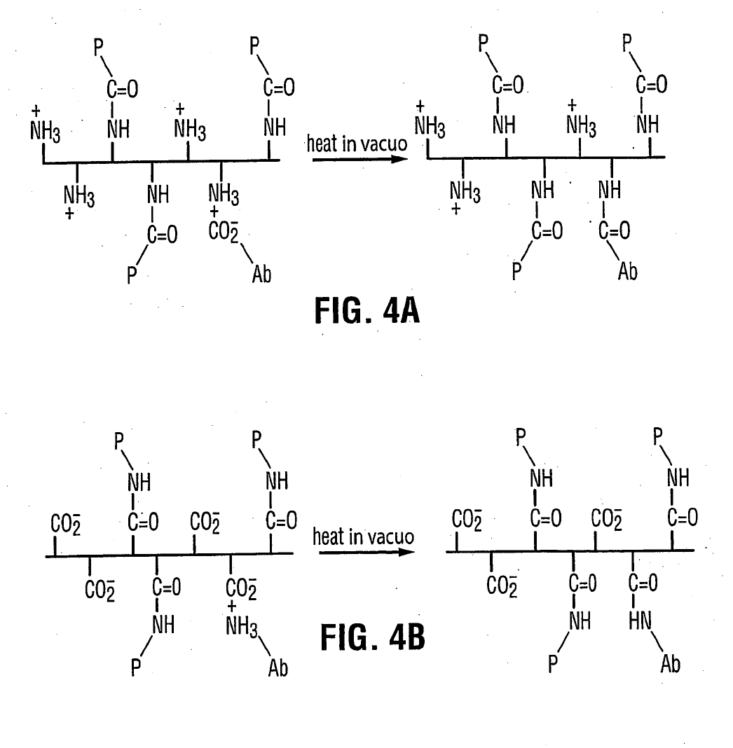

A reaction forming a cross-linked polylysine/antibody(Ab)/enzyme(P) complex of this kind is illustrated in FIGS. 4A. A similar reaction forming a polyglutamic acid/antibody(Ab)/enzyme(P) complex is shown in FIG. 4B. The antibody may be, for example, immunoglobulin. Upon heating under a vacuum, the mixture cross-links so that a complex is formed having both an enzyme and an antibody attached to a polyamino or polycarboxyl compound carrier. The present invention provides a convenient and reliable way of making such complexes. As previously noted, the protein polymer molecule is generally of large size (molecular weight) than both the enzyme and the antibody, thus creating a complex that can easily be isolated from the reaction mixture and employed in the manner explained.

The invention is illustrated in more detail by the following Examples. These Examples should not be viewed as limiting the scope of the present invention in any way.

In the Examples, the cross-linking procedure was carried out as follows.

Materials

Bovine pancreatic ribonuclease A (Type I-A), lysozyme, poly-D-lysine, poly-D-glutamic acid, and D(+)-trehalose were purchased from Sigma-Aldrich. All other chemicals, reagents and solvents were high purity preparations obtained from reliable commercial sources.

In Vacuo Cross-Linking Procedure

Lyophilized protein was obtained from a supplier, reconstituted in distilled water to a concentration of 10 mg/ml, and the pH of the solution was adjusted to 7.0 with 1 N NaOH. The protein solution was placed in a glass tube and lyophilized. These glass tubes were sealed under a vacuum of approximately 50 to 10 milli-torr and then placed in an oven at temperatures ranging from 50-120° C. for a minimum duration of 24 h. The vacuum was released and the protein sample reconstituted with 0.2 M Na₂HPO₄ and 0.15 M NaCl at pH 6.55 to give a final protein concentration of 10 mg/ml.

In some cases, the protein reconstituted in distilled water (dH₂O) instead of buffer to a concentration of 10 mg/ml, an aliquot was removed, and then the solution was re-lyophilized and heated again under vacuum at high temperatures for an additional 24 h. After four successive cycles of lyophilization, heating, and reconstitution, the final lyophilized protein sample was reconstituted with 0.2 M Na₂HPO₄ and 0.15 M NaCl at pH 6.55 to give a final protein concentration of 10 mg/ml.

### EXAMPLE 1

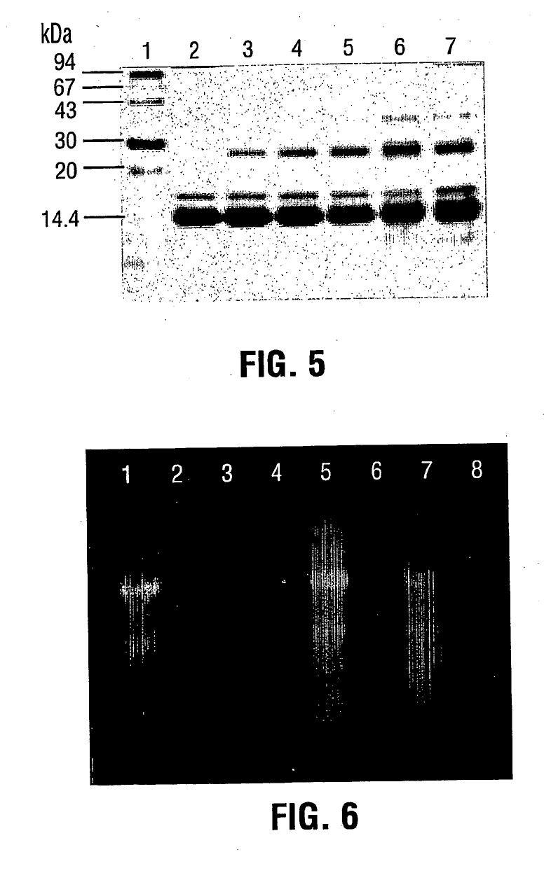

RNase A (LpH 7.0) was subjected to successive cycles of lyophilization, heating to 85° C. in vacuo for 24 h, and reconstitution. After each cycle, the product was subjected to SDS-PAGE, and the results are shown in FIG. 5, which shows seven lanes, as follows (the total protein load per lane was 20 μg):

Lane 1—a low range molecular weight marker.

Lane 2—lyopholized RNase A without heating in vacuo.

Lane 3—lyophilized RNase A heated for 24 hours in vacuo (cycle 1).

Lane 4—lyophilized RNase A heated for 48 hours in vacuo (cycle 2).

Lane 5—lyophilized RNase A heated for 72 hours in vacuo (cycle 3).

Lane 6—lyophilized RNase A heated for 84 hours in vacuo (cycle 4).

Lane 7—lyophilized RNase A heated continuously for 96 hours in vacuo with only one reconstitution.

The plate shows the monomer at 14.4 kDa, and the increasing development with time of a dimer at just below 30 kDa, as well as a trimer at about 43 kDa, etc. A strong band with an apparent molecular mass of ca. 28 kDa, expected for the RNase A dimer, becomes evident after only one heating period of 24 h in vacuo, and intensifies as the heating time increases to 96 h. Maximum dimer formation was obtained after 96 h of heating in vacuo, which is similar also for Lysozyme (see Example 2) as well as all other proteins tested. Size-exclusion chromatography of RNase A cross-linked products indicated that the total yield of the RNase A dimer was approximately 30% by weight of the total protein treated. Other tests showed that, when either the amino groups or the carboxyl groups were modified, by reductive methylation or amidation, respectively, in vacuo cross-linking of the lyophilized protein did not occur, thus confirming the involvement of these groups in the formation of the dimer.

This confirms that polymerization takes place under the indicated conditions.

### EXAMPLE 2

RNase A In-Gel Activity Assay

Cross-linked RNase A products were tested for catalytic activity using an RNA agarose gel-based assay (Leland et al., 1998; Gaur et al., 2001). Cross-linked RNase A products (2 ng) were incubated with 5 μg of total rat liver RNA in 100 mM Tris-HCl, pH 7.5, containing 10 mM DTT in a total reaction volume of 10 μl. Reaction was allowed to proceed for 10 min at 37° C. and was stopped by the addition of 1 μl of diethyl pyrocarbonate and followed by incubation on ice for 2 min. Samples were supplemented with 2 μl of RNA gel loading buffer (10 mM Tris-HCl, pH 7.5, 50 mM EDTA, glycerol (30% v/v), xylene cyanol FF (0.25% w/v), and bromophenol blue (0.25% w/v)) before loading onto 1.5% agarose gel containing 2% formaldehyde and 0.05 M ethidium bromide. RNA gel electrophoresis is shown in FIG. 5. The degradation of total RNA is visualized as all three un-nhibited RNase A species (native, monomer, and dimer). The inhibited native and monomer RNase A shows no degradation of RNA; however, the covalent RNase A dimer is not inhibited by the ribonuclease inhibitor and RNA is degraded.

FIG. 6 shows the results of an RNA in-gel RNase A activity assay. This shows that cross-linked RNase A dimer retains the enzymatic activity of the monomeric RNase A. In each reaction, 5 mg total rat liver RNA in 100 mM Tris buffer was incubated for 10 min at 37° C. with or without the enzyme, RNase A, and the inhibitor, cRI. An aliquot of the reaction mixture was then loaded onto a standard agarose gel and the RNA visualized by ethidium bromide staining as per the standard methodology.

Lane 1—total RNA control where 5 mg total rat liver RNA was incubated, no enzyme has been added.

Lane 2—2 ng of RNase A as supplied from a commercial source incubated with 5 mg total rat liver RNA.

Lane 3—2 ng of treated RNase A monomer as collected from FLPC incubated with 5 mg total rat liver RNA;

Lane 4—2 ng of treated RNase A dimer as collected from FPLC incubated with 5 mg total rat liver RNA;

Lane 5—a total RNA control; again, no enzyme has been added but 20 units of the RNaseA inhibitor, cRI is present.

Lane 6—2 ng of RNase A as supplied from a commercial source incubated with 5 mg of total RNA and 20 units of cRI;

Lane 7—2 ng of treated RNase A monomer incubated with 5 mg of total RNA and 20 units of cRI;

Lane 8—2 ng of treated RNase A dimer incubated with 5 mg of total RNA and 20 units of cRI.

This gel plate demonstrates that the dimer of RNase A exhibits activity similar to the monomer and that it is less susceptible to inhibition by a conventional inhibitor of the RNase A enzyme. This latter result confirms the structural integrity of the dimer formed under the indicated conditions. Electrospray TOF mass spectrometry data confirmed the presence of RNase A dimer corresponding to twice the molecular mass of native RNase A minus the loss of a water molecule.

RNase A dimer having one or more zero-length cross-links is believed to be a novel product with useful properties. The covalent in vacuo cross-linked RNase A dimer is approximately twice as active compared to native RNase A based on the respective catalytic activities per monomeric unit toward dsRNA in a Kunitz based assay (Kunitz, M. 1946. A Spectrophotometric Method for the Measurement of Ribonuclease Activity. J. Biol. Chem. 164. 563-568).

### EXAMPLE 3

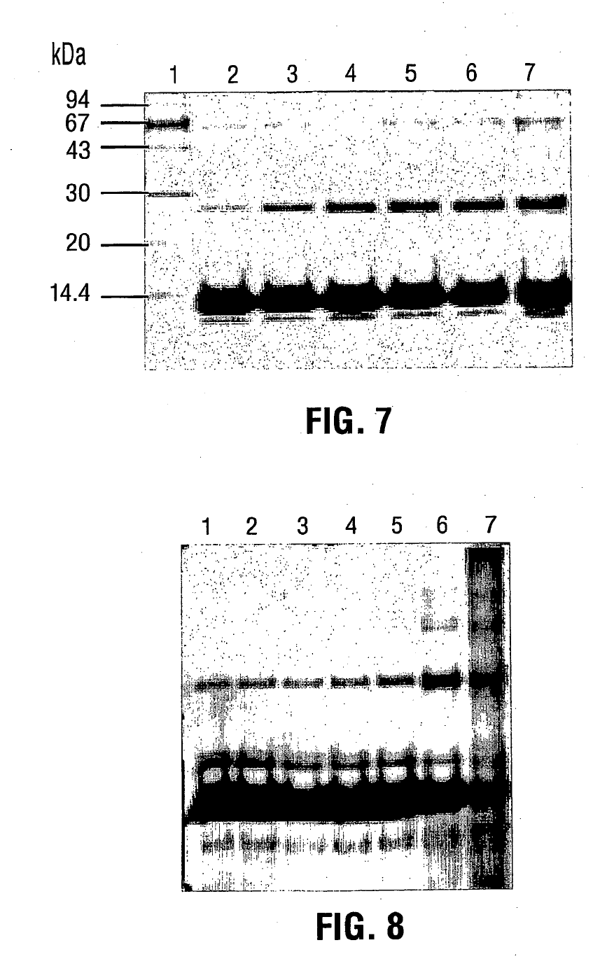

Lysozyme (LpH 7.0) was subjected to successive cycles of solubilization of 10 mg of the lysozyme at pH 7.0, lyophilization, then heating to 850C in vacuo for 24 h, and reconstitution. After each cycle, the product was subjected to gel electrophoresis, and the results are shown in FIG. 7, which shows seven lanes. The total protein load per lane was 20 μg.

Lane 1—low range molecular weight marker.

Lane 2—lyophilized lysozyme without heating in vacuo.

Lane 3—lyophilized lysozyme heated for 24 hours in vacuo (cycle 1).

Lane 4—lyophilized lysozyme heated for 48 hours in vacuo (cycle 2).

Lane 5—lyophilized lysozyme heated for 72 hours in vacuo (cycle 3).

Lane 6—lyophilized lysozyme heated for 84 hours in vacuo (cycle 4).

Lane 7—lyophilized lysozyme continuously heated for 96 hours in vacuo with only one reconstitution.

The plate shows the presence of a monomer (broad band at bottom of each lane), and the increasing development with time of a dimer (band of increasing height midway up each lane).

This confirms that dimerization takes place under the indicated conditions.

### EXAMPLE 4

The Effect of Heating Temperature in the In Vacuo Cross-linking Procedure

RNase A solutions of 2.5 mg/ml at pH 7.0 adjusted with IN NaOH were placed into glass vessels and sealed under vacuum. Each sample was heated at a different temperature ranging from 20° C. to 150° C. for 48 h. After the heating period, the samples were reconstituted in water and the cross-linked products (15 μg) were visualized on SDS-PAGE electrophoresis as shown in FIG. 8. It appears that heating temperatures between 100-120° C. generate the highest yield in dimer formation.

In FIG. 8:

Lane 1—sample in vacuo cross-linked at 23° C.;

Lane 2 is the trace of a sample in vacuo cross-linked at 40° C.;

Lane 3 is the trace of a sample in vacuo cross-linked at 55° C.;

Lane 4 is the trace of a sample in vacuo cross-linked at 70° C.;

Lane 5 is the trace of a sample in vacua cross-linked at 95° C.;

Lane 6 is the trace of a sample in vacuo cross-linked at 120° C.; and

Lane 7 is the trace of a sample in vacuo cross-linked at 150° C.

### EXAMPLE 5

The Effect of PH, Counter Ions, or Excipients

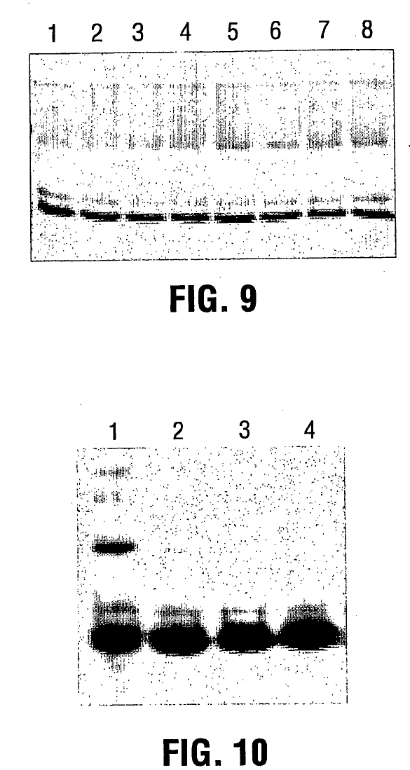

RNase A solutions (10 mg/ml) at pH values varying from 3.0 to 10.0 were prepared by the addition of 1 N NaOH or 1 N HCl with a micro-syringe, as required. The protein solutions were lyophilized and subjected to the in vacuo cross-linking procedure. A 10 μg sample of the treated protein was subjected to SDS-PAGE and the results are shown in FIG. 9. It was found that neutral to slightly alkaline pH values, i.e. pH 7.0-9.0, favor the formation of dimer.

In FIG. 9:

Lane 1—sample at pH 3;

Lane 2—sample at pH 4;

Lane 3—sample at pH 5;

Lane 4—sample at pH 6;

Lane 5—sample at pH 7;

Lane 6—sample at pH 8;

Lane 7—sample at pH 9; and

Lane 8—sample at pH 10.

### EXAMPLE 6

RNase A solutions (10 mg/mi) were also prepared in the presence of different cations by the addition of excess LiCl, NaCl or CsCl followed by dialysis against distilled water. Samples were treated as described above except that the pH was adjusted to 7.0 with 1 N LiOH 1 N NaOH, or 1 N CsOH, as appropriate. The effect of differing the counter ion did not change the extent of cross-linking and therefore the results are not shown.

### EXAMPLE 7

A solution of RNase A (10 mg/ml at pH 7.0) was lyophilized in the presence of D-trehalose at w/w ratios of protein/trehalose of 5:1, 1:1, and 1:5 and then subjected to the in vacuo cross-ling procedure for 96 h On completion, the excess trehalose was removed by dialysis. The SDS electrophoresis of 20 μg samples of the cross-linked products is shown in FIG. 10. As the amount of trehalose present in the lyophilized sample increases, the amount of RNase A dimer produced decreases. At a 1:1 w/w ratio, trehalose appears to prevent any dimer formation, as only a trace of dimer similar to that observed in untreated samples is present. In the experience of the inventors, not all excipients added to the protein solution prior to lyophilization and heating are equally effective in inhibiting the cross-lining reaction.

In FIG. 10:

Lane 1—RNase A alone cross-linked in vacuo;

Lane 2—RNase A and trehalose in 5:1 (w/w) ratio, cross-linked in vacuo;

Lane 3—RNase A and trehalose in a 1:1 (w/w) ratio, cross-linked in vacuo; and

Lane 4—RNase A and trehalose in a 1:5 (w/w) ratio, cross-linked in vacuo.

### EXAMPLE 8

Heterogeneous Cross-Linking In Vacuo

A solution containing RNase A and lysozyme in equal amounts (10 mg/ml) was prepared and the pH was adjusted to 7.0 with 1 N NaOH before lyophilization. The in vacuo procedure was carried out for 48 h on the mixture of these two proteins as previously described. The SDS electrophoresis analysis of the cross-linked products (15 μg) is shown in FIG. 11. Three bands are visible corresponding the cross-linked dimeric RNase, cross-linked dimeric lysozyme and the heterogeneously cross-linked lysozyme/RNase product.

In FIG. 11:

Lane 1—RNase A (pH 7.0) alone, cross-linked in vacuo for 48 h;

Lane 2—lysozyme (pH 7.0) alone, cross-linked in vacuo for 48 h; and

Lane 3—RNase A (pH 7.0) and lysozyme (pH 7.0) co-lyophilized and cross-linked in vacuo for 48 h.

### EXAMPLE 9

Poly-D-lysine (Mᵣ ˜340 000) or poly-D-glutamate (Mᵣ ˜32 000) was mixed with RNase A in solution in a 5:1 w/w (protein/polymer) ratio. After adjusting the pH to 7.0, the mixture was lyophilized and subjected to the in vacuo cross-linking procedure (refer to FIG. 1A and FIG. 2.). The cross-linked mixture was then separated via size exclusion FPLC chromatography and the high molecular weight fractions were tested for ribonuclease activity and were shown contain RNase A activity, which implies successful cross-linking of RNase A to protein polymer.

### EXAMPLE 10

Detection and Quantification of Cross-Linked Protein

Cross-linked products were detected by SDS-PAGE, using the BioRad™ mini protean II electrophoresis system. Protein (5-20 μg) was loaded onto a 16.5% Tricine™ SDS-polyacrylamide gel. After electrophoresis at 130 V for 90 min, the gel was stained with Coomassie Brilliant Blue G250. The relative amount of protein present in each band was determined using the pixel counting application in ImageQuaNT™ 5.1 (Molecular Dynamics).

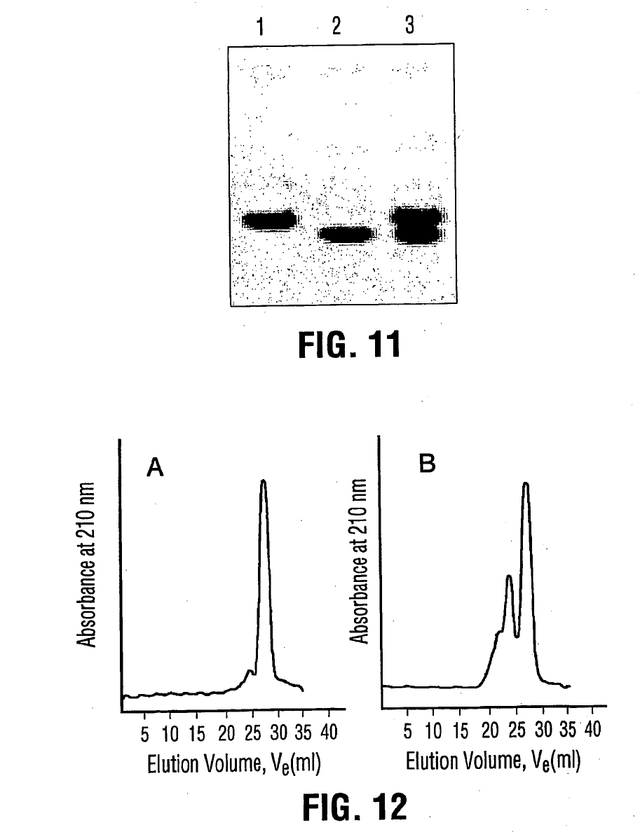

Size-exclusion chromatography was carried out using two Superdex™ G75 HR 10/30 columns (Amersham-Pharmacia) attached, in tandem, to a Pharmacia™ FPLC system with detection at 210 nm. Mobile phase (0.2 M Na₂HPO₄ and 0.15 M NaCl at pH 6.55 at 4° C.) was used at a flow rate of 0.05 ml/min. In general, 0.5 ml fractions were collected. Molecular weight standards (phosphorylase b, 94 kDa; bovine serum albumin, 67 kDa; ovalburnin, 43 kDa; bovine erythrocytes carbonic anhydrase, 29 kDa; trypsin inhibitor, 20.1 kDa; a-lactalbumin, 14.4 kDa) used in column calibration were purchased from Amersham Pharmacia Biotech. Pooled fractions containing RNase A dimer were concentrated to 0.5 ml using Centriprep™ (Amicon) 3,000 molecular weight cut-off concentrators. The size exclusion FPLC chromatogram of the in vacuo cross-linked RNase A mixture is shown in FIG. 12 (Trace A is for RNase A lyophilized at pH 7.0 with no cross-linking; Trace B is RNase A lyophilized at pH 7.0 and then cross-linked in vacuo). RNase A dimer yields varied from 20-30% of the total treated protein depending on the length of the heating period. The elution profile of the RNase A heated in vacuo for 96 h (FIG. 10B) has peaks with molecular masses of 28 000 Da, and 25 14 000 Da, corresponding to the RNase A dimer and monomer, respectively. From the total area under the peaks, the amount of dimer present was found to be approximately 30% of the total protein, in agreement with the estimate by pixel counting of the gel photographs.

### EXAMPLE 11

Chemical Modification of Proteins

Dimethylation of amino groups was carried out on RNase A (15 mg) according to the procedure described by Means and Feeney (1971). Excess reagent was removed by dialysis.

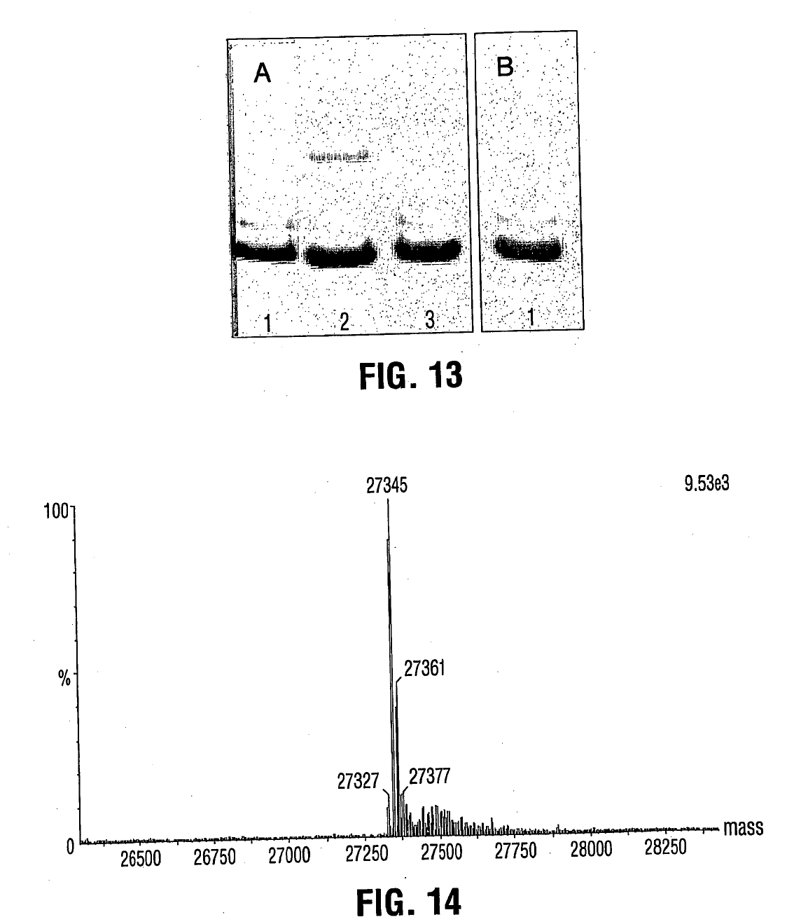

Amidation of carboxyl groups was carried out on a solution of RNase A (15 mg/ml in a total reaction volume of 1 ml) in 1.33 M glycinamide at pH 4.75 with activation by carbodiimide as describe in Means and Feeney (1971). On completion, excess reagent and by-products were removed by dialysis and the modified proteins were lyophilized and heated in vacuo under the same conditions as the unmodified protein. In both cases, no dimerization was observed as shown in FIG. 13.

In FIG. 13, the total protein loaded per lane was 10 micrograms:

A:

Lane 1 shows RNase A lyophilized at pH 9.0 with no in vacuo treatment;

Lane 2 shows RNase A lyophilized at pH 9.0 and heated in vacuo;

Lane 3 shows reductively methylated RNase A lyophilized at pH 9.0 and heated in vacuo for 48 h;

B:

Lane 1 shows RNase A with amidated carboxyl groups lyophilized at pH 7.0 and heated in vacuo for 48 h.

### EXAMPLE 12

Mass Spectrometric Analysis

A deconvoluted nanospray mass spectrum of the RNase A dimer produced by in vacuo cross-linking (FIG. 14) was obtained using a Micromass Q-Tof™ mass spectrometer. The MS data was deconvoluted using MaxEnt1™ software to provide the singly charged average masses. The RNase sample was purified by standard ZipTip methodology. Analytes were eluted with 75% methanol/25% water/0.2% formic acid, the sample was centrifliged (6000 rpm for 2 minutes) and then 2 μl was loaded into a gold coated nanospray needle (New Objectives Picotip). The key variable MS voltages include: capillary (+950 V), cone (+47 V), and RF lens 1.05; the source temperature was 80° C., and the data for each scan was collected for 5 seconds over the range 400 to 2500 Da, using aNaTFA solution for external calibration The major peak in the spectrum occurs at 27345 mass units corresponding to twice the mass of the monomer (13682±1 mass units) minus 18 mass units, i.e. the loss of one water molecule, showing that only one amide crosslink is present in the dimer. There is also a trace amount of a dimer peak at 27327 mass units resulting from the loss of two water molecules and the formation of two amide cross-links.

### EXAMPLE 13

Kunitz Ribonuclease A Activity Assay

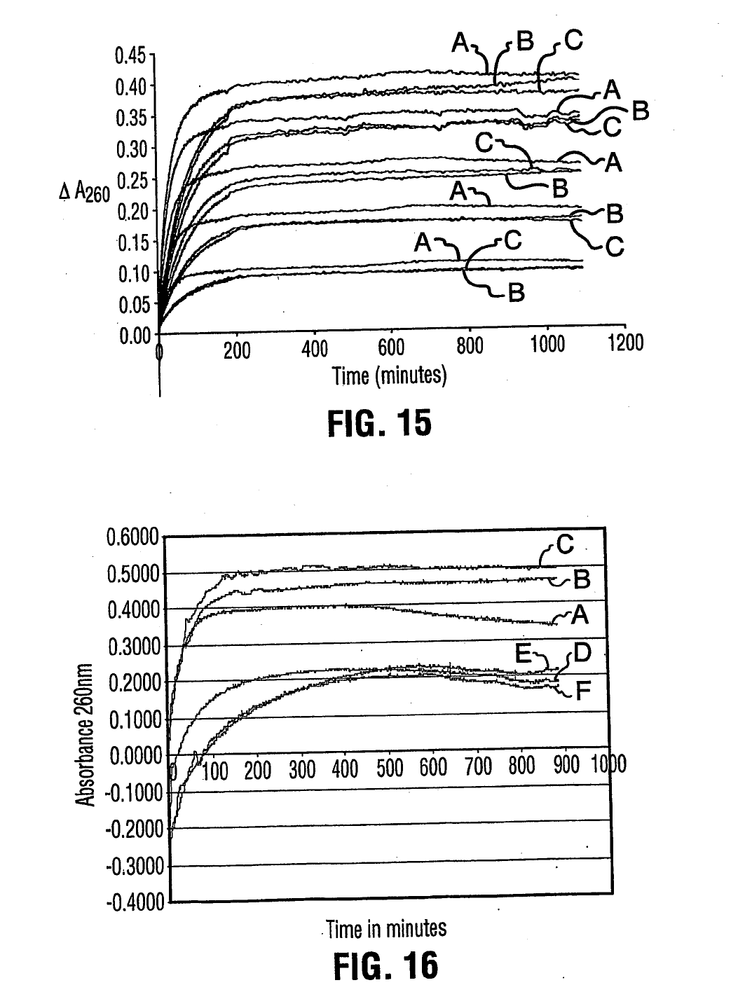

RNase A enzymatic activity was determined by quantifying rates of poly adenosine-poly cysteinic acid RNA substrate degradation over time spectrophotometrically as shown in FIG. 15. This plots the change in absorbance at 260 nm over time of enzymatic activity of non-treated RNase A (wild type) (plots C in the Figure), monomeric RNase A (lots B in the Figure), and in vacuo cross-linked dimeric RNase A (plots A in the Figure) in the presence of 20, 40, 60, 80 or 100 micrograms/mL poly(A).poly(U), showing the progression of the increase in absorbance at 260 nm over 18 hours of reaction The assay used is a modification of a method developed by Kunitz (reference to follow). One Kunitz unit of activity corresponds to an initial increase of absorbance at 260 nm of 100% per minute of the total measurable increase in absorbance measured after completion of the reaction (refer to equation 1).

$${U}_{Kwitz} = \frac{ⅆ A / ⅆ {t}_{\cdot initial}}{( {A}_{f} - {A}_{o} )} . \qquad Equation 1$$

The change in initial absorbance (dA/dt) divided by the difference of final (A_{f}) and initial (A₀) absorbance values at 260 nm was determined and enzymatic velocity (Vₒ) was calculated by multiplying the Kunitz activity by the initial substrate concentration [Sₒ], as shown in equation 2.

$${V}_{o} = \frac{ⅆ A / ⅆ t \cdot initial}{( {A}_{f} - {A}_{o} )} \cdot [ {s}_{o} ] . \qquad Equation 2$$

Enzyme and substrate solutions are prepared in Kunitz buffer (0.15M NaCl, 0.015M citrate at pH 7.4) and the reaction takes place in a 96-well microtitre plate. For each sample, tested 160 μL of substrate solution at 5/4 of the desired final concentration (the standard concentration is 80 μg/mL) are pipetted into a well in a 96 well UV-transparent flat bottom acrylic plate. 40 μL of enzyme solution at 5 times the desired concentration (the standard concentration is 50 μg/mL) are then added, holding the pipet tip under the surface of the already present mixture. Readings of the absorbance at 260 nm are taken during three hours at 1 minute intervals, then during 15 hours at 5 minute intervals on a Tecan SPECTRAFluorPlus multifunction microplate reader. In order to measure multiple samples separately, the substrate and enzyme solutions are first pipetted in excess (respectively 200 μL and a minimum of 100 μL per sample) into a sterile 96 well culture plate. The solutions are then transferred into the UV plate using multiple pipettors and the microplate reader monitors the rate of substrate degradation over time.

The value of k_{cat}/K_{M} for the cleavage of poly adenosine—poly cysteinic acid RNA substrate by wild type RNase A, RNase A in vacuo cross-linked dimer were then determined by the slope of Michaelis-Menton plots Vₒ versus [Sₒ], where the slope equals enzyme concentration [E]* k_{cat}/K_{M}. Results reported in Table 1.

TABLE I
k_{cat}/K_{M} and specific activity values for wild type, monomeric and
in vacuo cross-linked dimeric RNase A

| | k_{cat}/K_{M} | Specific Activity |
|---|---|---|
| | (min⁻¹*mg⁻¹ protein*mL) | (units Kunitz/mg protein) |
| Monomer | 0.42 ± 0.01 | 0.41 ± 0.02 |
| Dimer | 0.78 ± 0.03 | 0.83 ± 0.05 |

### EXAMPLE 14

Ribonuclease Inhibition Studies

Ribonuclease A activity was analyzed in the above Kunitz assay with the presence of anti-RNase A inhibitor (Ambion). By definition, 1 unit of inhibitor is that amount required to inhibit 50% of the activity of 5 ng of RNase A activity. Enzyme concentrations of 50 μg/ml were used which required 1000 U of anti-RNase for 50% inhibition. Substrate concentrations were held constant at 20 μg, for each enzyme assayed, namely, RNase A monomer and the in vacuo cross-linked dimer. The Kunitz plot is shown in FIG. 16. The dimer appears not to be inhibited by anti-RNase to the same degree as the monomeric RNase A. FIG. 16 shows a Kunitz ribonuclease inhibited activity assay plotting the change in absorbance at 260 nm over time of the enzymatic activity of monmeric RNase and in vacuo cross-linked dimeric RNase A in the presence of 20 micrograms poly(A).poly(U) and 2000 U or 3000 U of anti-RAase A inhibitor: progression of the increase in the absorbance at 260 nm over 18 hours of reaction.

In FIG. 16, the various plots are identified by letters A through F. These plots represent the following:

Plot A: Dimer with 20 μg of substrate

Plot B: Dimer with 20 μg of substrate and 2000 u of I

Plot C: Dimer with 20 μg of substrate and 3000 u of I

Plot D: Monomer with 20 μg of substrate

Plot E: Monomer with 20 μg of substrate and 2000 u of I

Plot F: Monomer with 20 μg of substrate and 3000 u of I.

### EXAMPLE 15

Immobilization of Proteins on Solid Supports

Chromatographic resins such as 4% beaded agarose and HyperD® ceramic beads derivitized with D-lysine (both purchased from Sigma-Aldrich) were resuspended in a alkaline phosphatase solution of 5 mg/ml, pH was adjusted to 7.0 with 1.0 N NaOH, then subjected to the in vacuo cross-linking procedure as previously described. After treatment, the resin was washed several times with the enzyme dilution buffer (0.1% w/v MgCl₂, 0.1% w/v ZnCl₂, 10% v/v glycerol, in 25 mM glycine at pH 9.6), then 5 ml of resin was packed into a small gravity flow column. The 3.9 mM p-nitrophenyl phosphate substrate solution in 25 mM glycine at pH 9.6 was then pumped through the column at 1 ml/min and 0.5 ml fractions were collected and absorbance at 405 nm was monitored to detect the presence of p-nitrophenol, the enzymatic product.

Glass beads (0.5 mm in diameter) were derivatized with 3-aminopropyltrimethoxysilane according to the method of Weetall. (H. H. Weetall, Nature, 223, 959 (1969)) attaching a propyl silica amine on the surface of the glass. Cytochrome c (˜100 μg) was dissolved in 200 μL distilled water (dH₂O) at pH 7 and was added to 25 mg of glass beads in an Eppendorf tube and was freeze-dried. The Eppendorf tube was placed in a vacuum at 50° C. for 15 hours. The glass beads were thoroughly washed with 2 mLs of phosphate buffered saline followed by 50 mLs of dH₂O. Activity was measured using the hydrogen peroxide/2,2′-azino-di-[3-ethyl-benzothiazoline-(6)-sulfonic acid assay which gives an oxidized colored product with an intense absorbance at 410 nm (Akasaka, R., Mashino, T., Hirobe, M., Arch. Biochem. Biophys., 301, 355-360 (1993)). The blank gave an absorbance reading of 0.004 units and two samples of cyctochrome c immobilized on the beads gave absorbance readings of 0.387 and 0.389 units showing that the immobilized cytochrome c is highly active.

1. A method of cross-linking molecules of poly (amino acid) compounds together or attaching such molecules to molecules of a polyamino or polycarboxyl compound, which comprises: lyophilizing a solution containing molecules of at least one poly (amino acid) compound, or at least one poly (amino acid) compound and a polyamino or polycarboxyl compound, thereby producing a lyophilized solid; maintaining the lyophilized solid under vacuum; heating the lyophilized solid maintained under vacuum to an elevated temperature effective to cause cross-linking or attachment of said molecules, thereby producing a reaction mixture comprising at least one cross-linked poly (amino acid) compound or at least one poly (amino acid) compound attached to said polyamino or polycarboxl compound; and cooling the reaction mixture and releasing the vacuum.

2. A method according to claim 1, characterized in that said solution has a pH prior to said lyophilizing at which amine groups of said molecules are protonated and carboxyl groups of said molecules are deprotonated.

3. A method according to claim 1, characterized in that said solution, prior to said lyophilizing, has a pH in the range of 4 to 10.

4. A method according to claim 1, characterized in that said solution, prior to said lyophilizing, has a pH in the range of 6 to 9.

5. A method according to claim 1, characterized in that said solution, prior to said lyophilizing, has a pH in the range of 7 to 9.

6. A method according to any one of claims 2, characterized in that said solution, when initially formed, has a pH differing from said pH prior to lyophilizing, and acid or base is added to said solution to provide said solution with said pH prior to said lyophilizing.

7. A method according to claim 1, characterized in that said heating of the lyophilized solid maintained under vacuum is carried out at a temperature in the range of 25 to150 C.

8. A method according to claim 1, characterized in that said heating of the lyophilized solid maintained under vacuum is carried out at a temperature in the range of 50 to150 C.

9. A method according to claim 1, characterized in that heating of the lyophilized solid maintained under vacuum is carried out at a temperature in the range of 70 to 100 C.

10. A method according to claim 1, characterized in that heating of the lyophilized solid maintained under vacuum is carried out at a temperature in the range of 80 to120 C.

11. A method according to claim 1, characterized in that heating of the lyophilized solid maintained under vacuum is carried out at a temperature in the range of 100 to120 C.

12. A method according to claim 1, characterized in that said heating of the lyophilized solid maintained under vacuum is carried out for a period of 1 to 96 hours.

13. A method according to claim 1, characterized in that said heating of the lyophilized solid maintained under vacuum is carried out for a period of 1 to 24 hours.

14. A method according to claim 1, characterized in that said at least one poly (amino acid) compound comprises a polypeptide.

15. A method according to claim 1, characterized in that said at least one poly (amino acid) compound comprises a protein.

16. A method according to claim 1, characterized in that said at least one poly (amino acid) compound comprises a complex protein.

17. A method according to claim 1, characterized in that said solution contains only one or more proteins.

18. A method according to claim 1, characterized in that said solution contains one or more proteins and a polyamino or polycarboxyl compound.

19. A method according to claim 1, characterized in that said at least one poly (amino acid) compound comprises a ribonuclease.

20. A method according to claim 19, characterized in that said ribonuclease is RNase A.

21. A method according to claim 1, characterized in that said at least one poly (amino acid) compound comprises a hemoglobin.

22. A method according to claim 1, characterized in that said solution contains one or more of an antibody and an enzyme, as well as a synthetic polyamine or a synthetic polycarboxyl compound.

23. A method according to claim 22, characterized in that said synthetic polyamine is polylysine.

24. A method according to claim 22, characterized in that said synthetic polycarboxyl compound is polyglutamic acid.

25. A method according to claim 1, characterized in that said solution contains at least one of alkaline phosphatase, human growth hormone, bovine serum albumin, beta-galactosidase, trypsin, chymotrypsin, Bt-toxin, cytochrome c and a de novo designed proteinMB-1 and variants thereof.

26. A method according to claim 1, characterized in that solution contains only one poly (amino acid) compound.

27. A method according to claim 1, characterized in that said solution contains two poly (amino acid) compounds.

28. A method according to claim 1, characterized in that said solution contains more than two poly (amino acid) compounds.

29. A method according to any one of claims 26, characterized in that said poly (amino acid) compound (s) is (are) a complex protein having biological activity in a living organism or biochemical activity on a non-living substrate.

30. A method according to claim 29, characterized in that said solution contains polylysine as a polyamino compound.

31. A method according to claim 30, characterized in said solution contains polyglutamic acid as a polycarboxyl compound.

32. A method according to claim 30, characterized in that molecules of said polylysine or said polyglutamic acid are attached to a solid surface.

33. A method according to claim 32, characterized in that said solid surface is a surface of glass or a synthetic polymer.

34. A method according to claim 1, characterized in that said solution contains a complex protein having biological activity in a living organism or biochemical activity on a non-living substrate, and a protein polymer having a plurality of unreacted carboxyl groups.

35. A method according to claim 34, characterized in that said protein polymer is polyglutamic acid.

36. A method according to claim 34, characterized in that said protein polymer is attached to a solid surface.

37. A method according to claim 36, characterized in that said solid surface is a surface of glass or a synthetic polymer.

38. A method according to claim 1, characterized in that said solution contains a synthetic compound having a plurality of unreacted amine groups or carboxyl groups and a protein having enzymatic activity to provide a cross-linked protein complex having several residues of said protein having enzymatic activity cross-linked to said synthetic compound.

39. A method according to claim 1, characterized in that said solution contains a cross-linked protein complex formed by a method of claim38 and an antigen or antibody protein.

40. A method according to claim 1, characterized in that said solution contains a cross-linked protein product having at least two protein residues, and at least one different protein.

41. A method according to claim 40, characterized in that said cross-linked protein product contains residues of at least two proteins directly covalently cross-linked together.

42. A process of directly attaching at least one protein molecule to a polycarboxyl or polyamino compound, which comprises lyophilizing a solution of at least one protein and at least one polyamino or polycarboxyl compound, thereby producing a lyophilized solid; maintaining the lyophilized solid under vacuum; heating the lyophilized solid maintained under vacuum to an elevated temperature effective to cause cross-linking of the protein and the polyamino or o polycarboxyl compound, thereby producing a product comprising a covalently cross-linked protein and polyamino or polycarboxyl compound; cooling the product and releasing the vacuum to produce a reaction mixture; and, if desired, isolating a cross-linked product from the reaction mixture.

43. A method of cross-linking protein molecules, which comprises lyophilizing a solution of at least one protein thereby producing a lyophilized solid, maintaining the lyophilized solid under vacuum, heating the lyophilized solid maintained under vacuum to an elevated temperature effective to cause cross-linking of said at least one protein, thereby producing a product comprising at least one cross-linked protein; and cooling the product and releasing the vacuum.

44. A method of forming a covalent bond between a molecule having at least one amino group and a molecule having at least one carboxyl group, characterized in that a solution containing said molecules is formed, said solution is lyophilized to form a lyophilized solid, maintaining said lyophilized solid under vacuum, heating the lyophilized solid maintained under vacuum to an elevated temperature effective to form said covalent bond, and releasing said vacuum
